(19) United States
(12) Patent Application Publication
(10) Pub. No.: US 2011/0160495 A1
(43) Pub. Date: Jun. 30, 2011

(54) SYNTHESIS OF HIGH MOLECULAR WEIGHT PRIMARY ALCOHOLS

(75) Inventors: NICOLE HASLING, DURANGO, CO (US); ELISE NAUGHTON, BLACKSBURG, VA (US); JAMES F. HURLEY, DURANGO, CO (US); ROBERT MILOFSKY, DURANGO, CO (US); BRUCE STEVENS, DURANGO, CO (US)

(73) Assignee: S&S Chemical, LLC, Durango, CO (US)

(21) Appl. No.: 12/912,136

(22) Filed: Oct. 26, 2010

Related U.S. Application Data
(60) Provisional application No. 61/255,049, filed on Oct. 26, 2009.

(51) Int. Cl. C07C 29/48

(52) U.S. Cl. 568/910.5; 568/912

(57) ABSTRACT

The present invention relates to methods for preparing high molecular weight aliphatic primary alcohol using hydroboration reactions.

### CROSS-REFERENCES TO RELATED APPLICATIONS

The present application claims priority to U.S. Provisional Application No. 61/255,049, filed Oct. 26, 2009, which is herein incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The synthesis of linear aliphatic primary alcohols by modification of Fischer-Tropsch catalysts has been carried out on an industrial scale since the mid 1920s. Linear aliphatic primary alcohols have also been synthesized with modified Ziegler-Natta catalysts on an industry scale since the mid 1970s. These high molecular weight primary alcohols are useful as additives in adhesives, paints, toners, personal care products and as precursors to esters and acids. Recently, C₂₄-C₃₀ alcohols have seen use in pharmaceutical compositions, foodstuffs, dietary supplements and may be effective at lowering cholesterol and consequently the risk of heart disease (see, Perez, P., U.S. Pat. No. 6,225,354).

During the past decade, numerous approaches to the synthesis of higher alcohols have been published. The bulk of these syntheses require expensive catalysts and most are not economically viable on a large scale.

Hydroborations using borane-tetrahydrofuran or borane-dimethyl sulfide (see, Huang, S. W.; Peng, W. L.; Shan, Z. X. and Zhao, D. J. New J. Chem., 2001, 25, 869-871), chloroborane adducts (see, Kanth, J. V. B.; Brown, H. C. J. Org. Chem., 2001, 66, 5359-5365) and transition metal catalyzed hydroboration reagents (see, Evans, D. A. and Fu, G. C. J. Org. Chem., 1990, 55, 2280-2282; Evans, D. A.; Muci, A. R. and Sturmer, R. J. Org. Chem., 1993, 58, 5307-5309) have generated significant interest in organic synthesis. Unfortunately, many of these hydroborating reagents are not commercially available or are not economically viable with respect to industrial-scale hydroboration-oxidation of alkenes. Clay and Vedejs (J. Am. Chem. Soc., 2005, 127, 5766-5767) have shown that commercially available pyridine borane can be activated by iodine in dichloromethane (generating PyBH₂I) to facilitate the hydroboration of 1-dodecene in 98% yield. While the reaction proceeds at room temperature, the toxic pyridine-borane is water reactive, limiting its utility for large-scale hydroboration-oxidation reactions. Recently, Huang et al. reported on the preparation and properties of sodium malonyloxyborohydride (SMB)—a monofunctional hydroborating agent. SMB was found to be an effective reagent for the one-pot hydroboration-oxidation of alkenes including 1-octene and 1-heptene. The hydroboration of terminal olefins was highly regioselective with primary alcohols obtained nearly exclusively (>97%) after oxidation with H₂O₂.

Despite the efforts above, there remains a need for the hydroboration-oxidation of higher molecular weight alkenes (including C₁₈, C₂₀, C₂₄, C₂₆, C₂₈, C₃₀ and higher alpha olefins), that is efficient, can be conducted on a commercial scale and avoids toxic side products. Surprisingly, the present invention fulfills this and others needs.

### BRIEF SUMMARY OF THE INVENTION

In one embodiment, the present invention provides a method for the preparation of a high molecular weight aliphatic primary alcohol. The method includes (a) contacting a high molecular weight α-olefin with a borohydride salt of the formula: M⁺B⁻¹HₙX₄₋ₙ and a malonic acid having the formula: HOC(O)—C(R^{a})(R^{b})—C(O)OH under conditions sufficient to form an intermediate product; and (b) contacting the intermediate product with an oxidizing agent to form the high molecular weight aliphatic primary alcohol. M⁺ is a cation selected from Li⁺, Na⁺, K⁺, Cs⁺ or N⁺R^{c}₄, wherein R^{c} is Et, Bu, benzyl, C₈₋₂₆ alkyl or C₈₋₃₀ alkyl. The subscript n is an integer from 1 to 4. Each X is independently H, —CN, or —OC(O)C₁₋₈ alkyl and R^{a} and R^{b} are each independently selected from the group consisting of H, CH₃, CH₂CH₃, i-Pr, i-Bu or t-Bu.

In another embodiment, the present invention provides a method for the preparation of a high molecular weight aliphatic primary alcohol. The method includes (a) contacting a high molecular weight α-olefin with a borohydride salt of the formula:

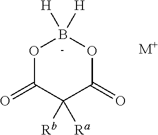

under conditions sufficient to form an intermediate product; and (b) contacting the intermediate product with an oxidizing agent to form the high molecular weight aliphatic primary alcohol. M⁺ is a cation selected from Li⁺, Na⁺, IC, Cs⁺ or N⁺R^{c}₄, wherein R^{c} is Et, Bu, benzyl, C₈₋₂₆ alkyl or C₈₋₃₀ alkyl. The subscript n is an integer from 1 to 4 and R^{a} and R^{b} are each independently selected from the group consisting of H, CH₃, CH₂CH₃, i-Pr, i-Bu or t-Bu.

### BRIEF DESCRIPTION OF THE DRAWINGS

FIG. 1 shows the ¹H NMR spectra of C₂₆₋₂₈ α-olefins.

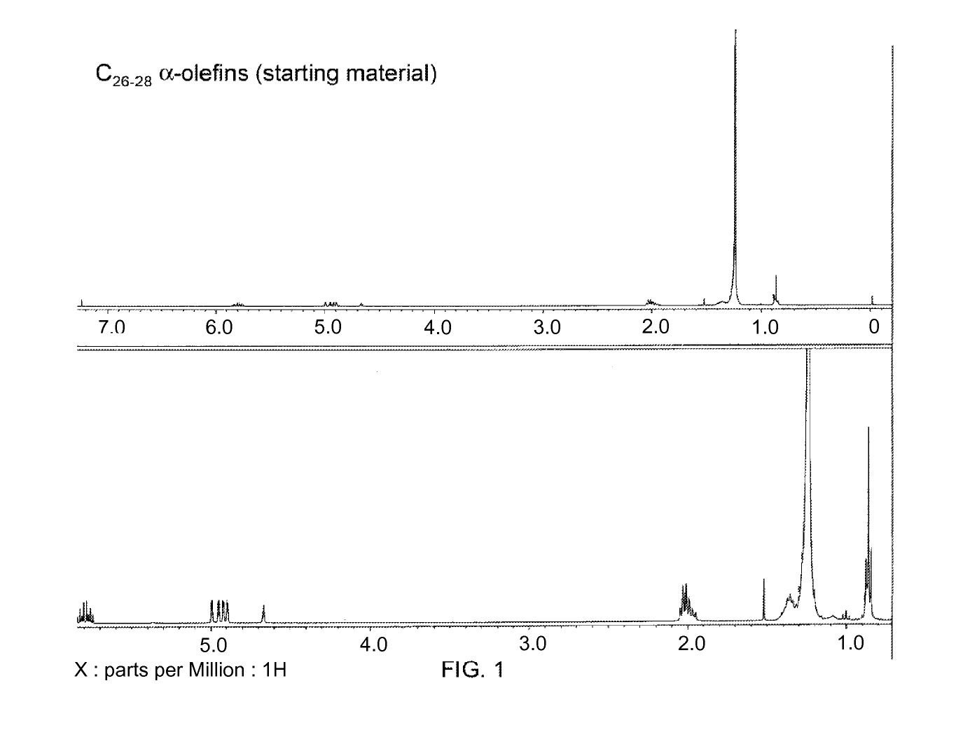

FIG. 2 shows the ¹H NMR spectra of purified C₂₆₋₂₈ alcohols obtained from the hydroboration reactions.

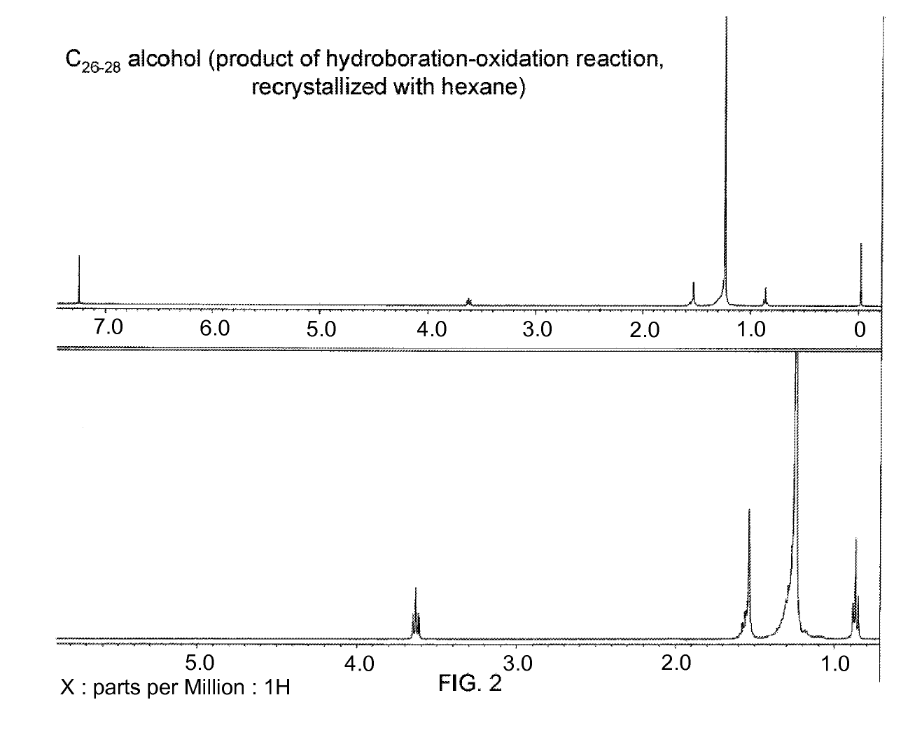

FIG. 3 shows a comparison of the chromatograms of the starting materials and the reaction product mixture following a hydroboration reaction. (a): 1-hexacosanol standard. (b): C₂₆₋₂₈ α-olefins starting material. (c): product mixture after the hydroboration reaction.

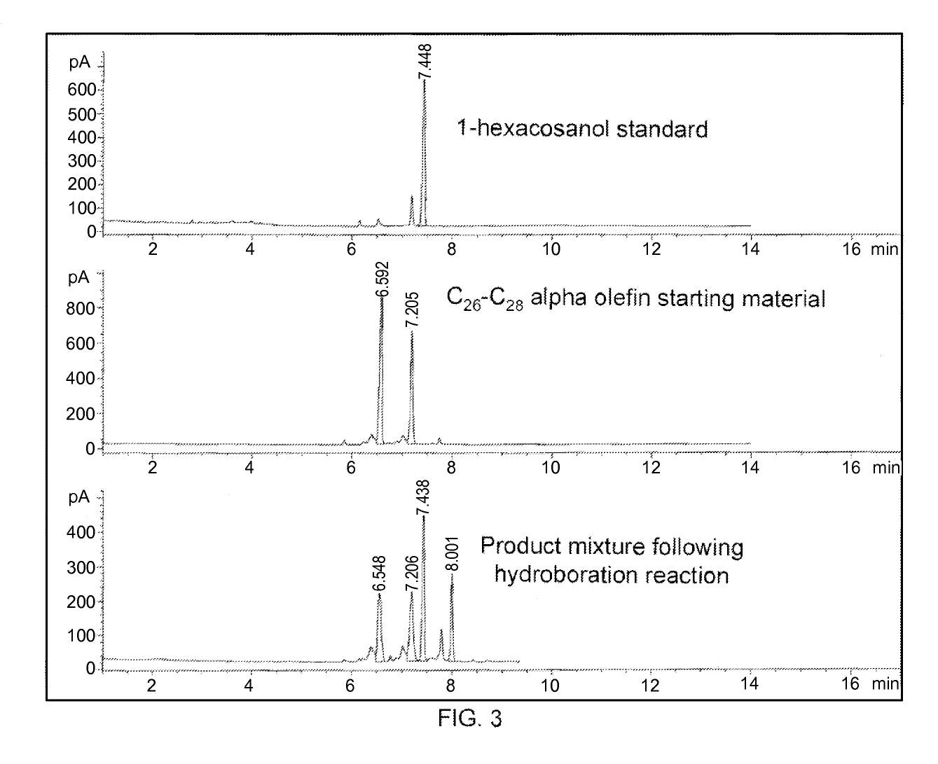

FIG. 4 illustrates a comparison of the chromatograms of the purified alcohol product and the crude hydroboration product mixture. (a): alcohol product purified by recrystallization. (b): crude hydroboration product.

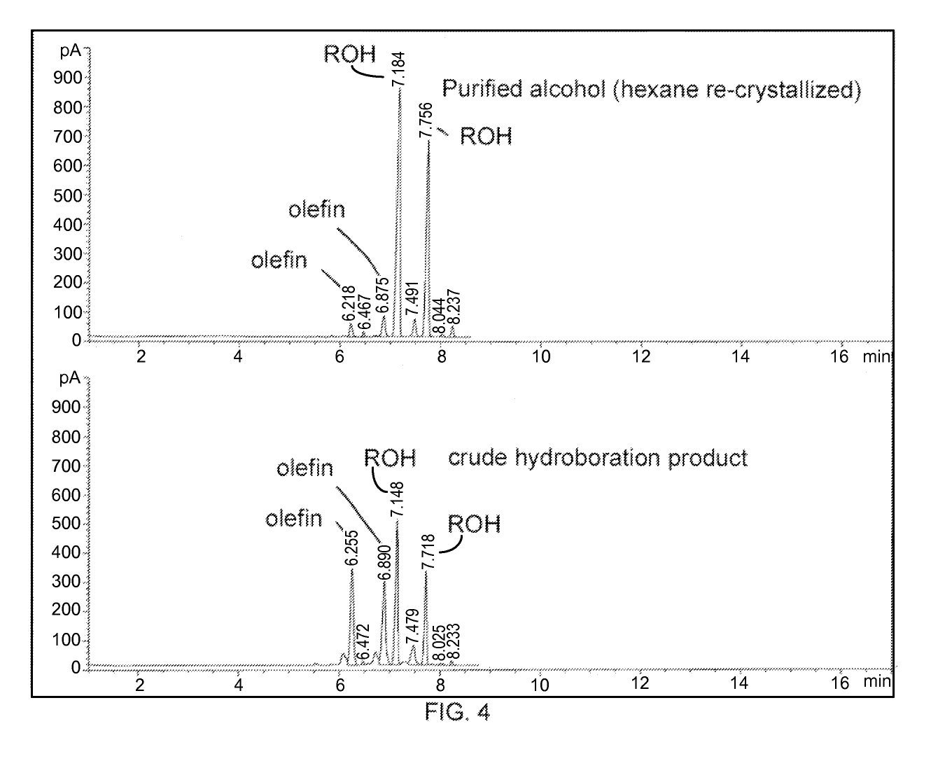

FIG. 5 shows a ¹H NMR spectrum of a C₁₈ α-olefin starting material.

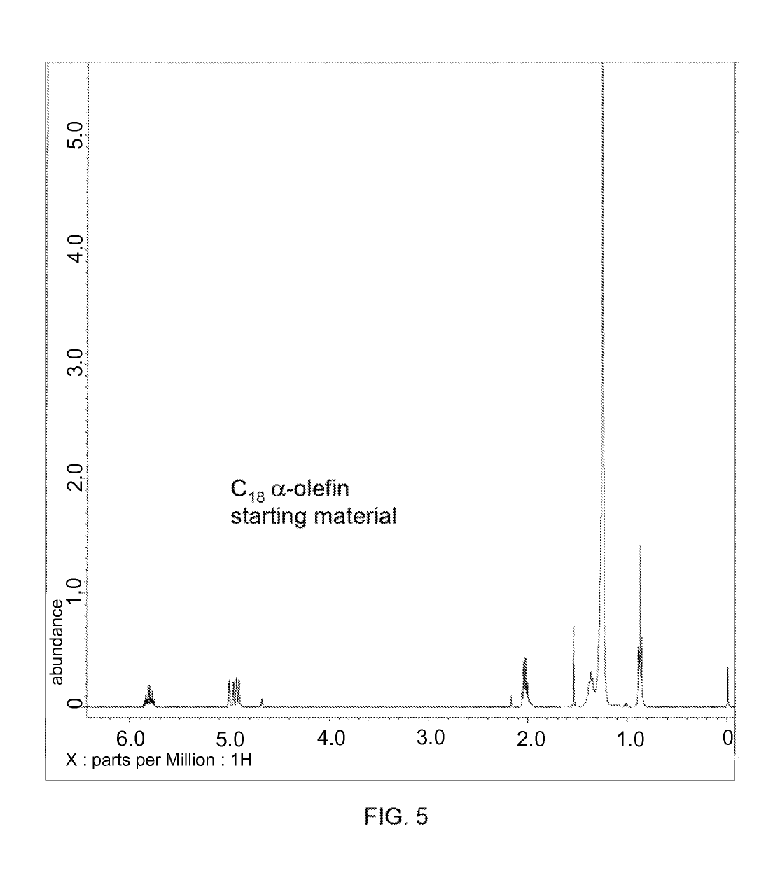

FIG. 6 provides a ¹H NMR spectrum of a 1-octadecanol standard.

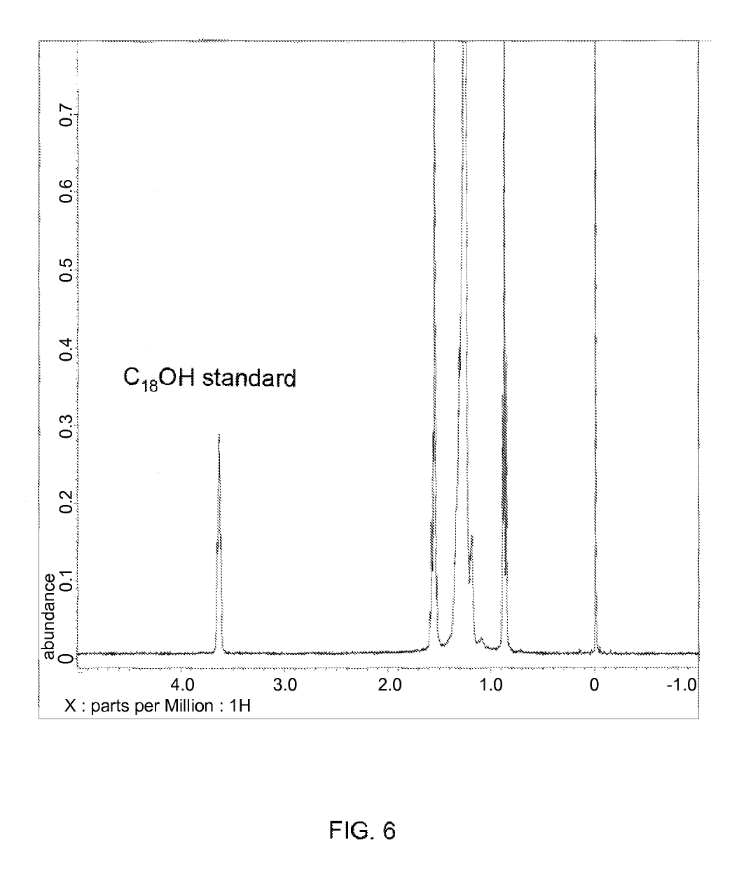

FIG. 7 shows a ¹H NMR spectrum of 1-octadecanol crude product obtained from a hydroboration reaction.

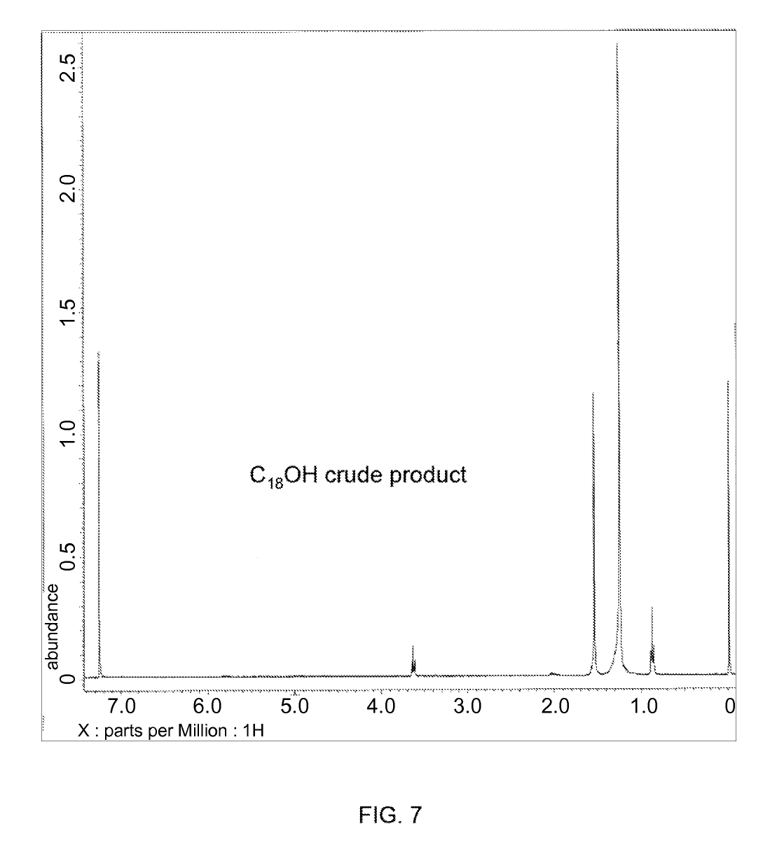

FIG. 8 depicts a gas chromatography spectrum of 1-octadecanol obtained from a hydroboration reaction.

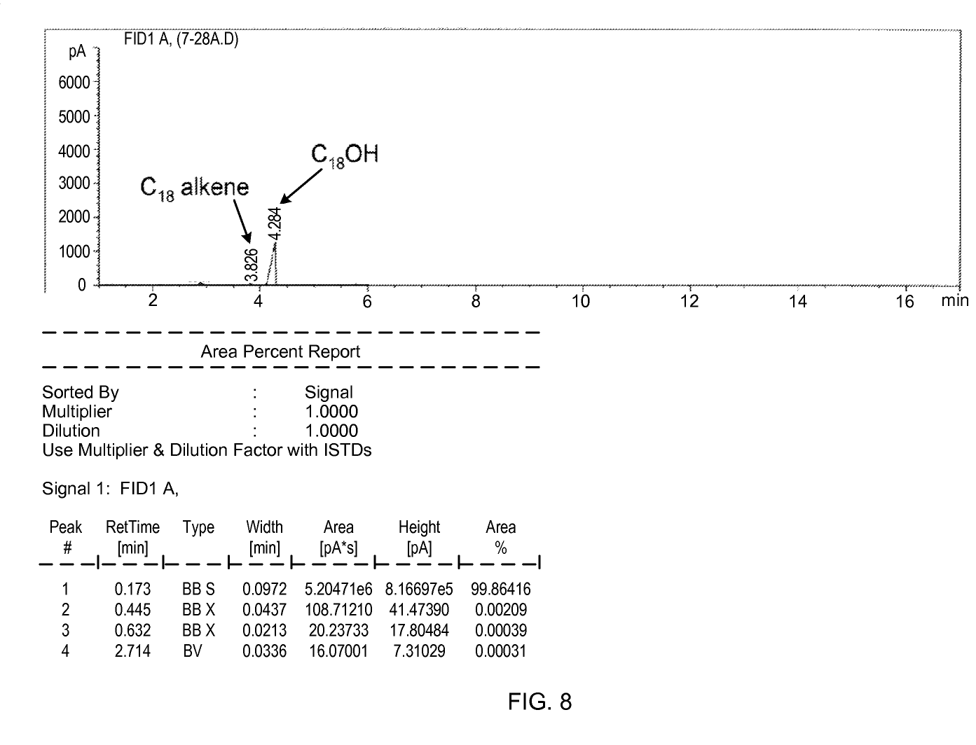

### DETAILED DESCRIPTION OF THE INVENTION

### I. General

The present invention relates to methods for preparing high molecular weight primary aliphatic alcohols from high molecular weight alkenes through hydroboration-oxidation reactions. Advantageously, the methods provide a commercially viable approach for the efficient synthesis of high molecular weight primary aliphatic alcohols that exhibit high regioselectivity.

### II. Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. When a prefix is not included to indicate the number of main chain carbon atoms in an alkyl portion, the radical or portion thereof will have 12 or fewer main chain carbon atoms or 8 or fewer main chain carbon atoms. For example, C₁₋₈alkyl refers to a hydrocarbon radical straight or branched having 1, 2, 3, 4, 5, 6, 7 or 8 carbon atoms and includes, but are not limited to, C₁₋₂alkyl, C₁₋₄alkyl, C₂₋₆alkyl, C₂₋₄alkyl, C₁₋₆alkyl, C₂₋₈alkyl, C₁₋₇alkyl, C₂₋₇alkyl and C₃₋₈alkyl.

The term “α-olefin” means an alkene where the carbon-carbon double bond starts at the alpha-carbon atom. Alpha-olefin is generally described by the formula: CH₂═CHR′, where R′ is an alkyl as defined herein, which can be optionally substituted with a functional group that is compatible with a hydroboration reaction condition. Non-limiting exemplary alkyl groups can have C₁₈, C₂₀, C₂₂, C₂₄, C₂₆, C₂₈, C₃₀ and higher carbon atoms and include linear or branched structures or a mixture thereof.

The term “high molecular weight α-olefin” means an α-olefin as defined herein containing at least 12 carbon atoms, preferably at least 16 carbon atoms, more preferably at least 20 carbon atoms. Non-limiting exemplary high molecular weight α-olefin have 18, 19, 20, 21, 22, 23, 24, 25, 26, 27, 28, 29, 30 and higher carbon atoms.

The term “high molecular weight aliphatic alcohol” means an aliphatic alcohol containing at least 12 carbon atoms, preferably at least 16 carbon atoms, more preferably at least 20 carbon atoms. Non-limiting exemplary high molecular weight aliphatic alcohols have 18, 19, 20, 21, 22, 23, 24, 25, 26, 27, 28, 29, 30 and higher carbon atoms.

The term “regioselectivity” as used herein refers to the ratio of the primary aliphatic alcohols versus the secondary aliphatic alcohols obtained during the hydroboration process.

The symbol [symbol] , whether utilized as a bond or displayed perpendicular to a bond, indicates the point at which the displayed moiety is attached to the remainder of the molecule.

### III. The Methods

In one aspect, the present invention provides a method for the preparation of high molecular weight aliphatic primary alcohols. The method includes contacting a high molecular weight α-olefin with a borohydride salt of the formula: M⁺B⁻HₙX₄₋ₙ and a malonic acid having the formula: HOC(O)—C(R^{a})(R^{b})—C(O)OH under conditions sufficient to form an intermediate product; and (b) contacting the intermediate product with an oxidizing agent to form the high molecular weight aliphatic primary alcohol. M⁺ is a cation selected from Li⁺, Na⁺, K⁺, Cs⁺ or N⁺R^{c}₄, wherein R^{c} is Et, Bu, benzyl, C₈₋₃₀ alkyl. The subscript n is an integer from 1 to 4. Each X is independently H, —CN, —SCN or —OC(O)C₁₋₈ alkyl. R^{a} and R^{b} are each independently selected from the group consisting of H, CH₃ and CH₂CH₃. The reaction can be carried out in situ to form the desired high molecular weight aliphatic primary alcohols. Generally, the reaction can be carried out at a temperature from about 22° C. to about 200° C. The reactants can be added in any order. In one embodiment, the intermediate product is formed by contacting a high molecular weight α-olefin with a borohydride salt of the formula: M⁺B⁻HₙX₄₋ₙ to form a reaction mixture, then contacting the reaction mixture with a malonic acid having the formula: HOC(O)—C(R^{a})(R^{b})—C(O)OH under suitable reaction conditions. The reaction mixture is typically formed in the presence of a solvent at an ambient temperature. In another embodiment, the reaction mixture is formed by mixing a borohydride salt of the formula: M⁺B⁻HₙX₄₋ₙ with a malonic acid having the formula: HOC(O)—C(R^{a})(R^{b})—C(O)OH. The intermediate product is formed by contacting a high molecular weight α-olefin with the reaction mixture under suitable reaction conditions. In yet another embodiment, the reaction mixture is formed by mixing a high molecular weight α-olefin with a malonic acid having the formula: HOC(O)—C(R^{a})(R^{b})—C(O)OH. The intermediate product is formed by contacting a borohydride salt of the formula: M⁺B⁻HₙX₄₋ₙ with the reaction mixture under a suitable reaction condition.

In a related aspect, the present invention provides a method for the preparation of a high molecular weight aliphatic primary alcohol. The method includes contacting a high molecular weight α-olefin with a borohydride salt of the formula:

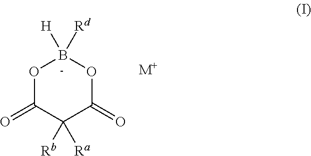

under conditions sufficient to form an intermediate product and contacting the intermediate product with an oxidizing agent to form the high molecular weight aliphatic primary alcohol. M⁺ is a cation selected from Li⁺, Na⁺, K⁺, Cs⁺ or N⁺R^{c}₄, wherein R^{c} is Et, Bu, benzyl or C₈₋₃₀ alkyl. R^{d} is H, CN, t-Bu or —OC(O)C₁₋₈alkyl. R^{a} and R^{b} are each independently selected from the group consisting of H, CH₃ and CH₂CH₃, i-Pr, i-Bu or t-Bu. R^{d} is preferably H, CN, —OAc or t-Bu. More preferably, R^{d} is H. R^{a} and R^{b} are preferably H. In a preferred embodiment, R^{a}, R^{b}) and R^{d} are H.

The starting material, high molecular weight α-olefins can be obtained commercially or prepared according to literature procedures. The high molecular weight α-olefins can generally be represented by the following formula: CH₂═CHR′, where R′ is an alkyl group having at least 10 carbon chain atoms, preferably at least 16 carbon chain atoms, more preferably at least 28 carbon chain atoms. In some embodiments, the R′ group has at least 38 carbon chain atoms. The alkyl group can be linear or branched. The starting material can be a mixture of linear or branched α-olefins. In certain instances, the α-olefins have between 15 and 20 carbon chain atoms. In other instances, the α-olefins have between 20 and 30 carbon chain atoms. Exemplary alkyl groups in the α-olefins can have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49 or 50 carbon atoms.

The high molecular weight α-olefin can present as a mixture of two or more high molecular weight α-olefins In one embodiment, the high molecular weight α-olefin is a mixture of C₂₆ to C₃₀ high molecular weight α-olefins. In another embodiment, the high molecular weight α-olefin is present as a mixture of C₁₂ to C₁₈ high molecular weight α-olefins. In yet another embodiment, the high molecular weight α-olefin is present as a mixture of high molecular weight α-olefins having 30 or more carbon chain atoms.

In some embodiments, the R′ group in the α-olefins can be further optionally substituted with one or more functional groups that are compatible with the reaction conditions, such as the hydroboration-oxidation conditions. Exemplary functional groups include, aryl, cycloalkyl, alkoxy, cycloalkoxy, aryloxy, arylalkoxy, dialkylaminocarbonyl, alkylarylaminocarbonyl, alkylcarbonylalkylamino, arylcarbonylalkylamino. In some embodiments, the R′ group can be optionally substituted with one or more functional groups selected from methoxy, ethoxy, benzyloxy, phenoxy, cyclopentoxy, cyclopentyl, cyclohexyl, phenyl, dimethylaminocarbonyl, dibenzylaminocarbonyl, methylphenylaminocarbonyl or benzylmethylaminocarbonyl.

Various borohydride salts can be used for the synthesis of high molecular weight aliphatic primary alcohols. In some embodiments, the borohydride salts can be represented by the following formulas: M⁺B⁻H₁X₃, M⁺B⁻H₂X₂, M⁺B⁻H₃X and M⁺B⁻H₄. In one embodiment, M⁺ is Li⁺, Na⁺K⁺ or Cs⁺. In another embodiment, M⁺ is N⁺R^{c}₄, wherein R^{c} is methyl, ethyl, propyl, butyl, isopropyl, pentyl, hexyl, heptyl, octyl, an aliphatic alkyl group having from 8 to 30 carbon atoms, or isomers thereof.

In some embodiments, the borohydride salts are M⁺B⁻H₄, M⁺B⁻H₃CN or M⁺B⁻H(OAc)₃, wherein M⁺ is Li⁺, Na⁺K⁺ or Cs⁺ or N⁺R^{c}₄, wherein R^{c} is as defined above.

The X group in the borohydride salts can be hydrogen, cyano, —SCN, C₁₋₄ alkyl or acyloxy. In one embodiment X is H. In another embodiment, X is —CN, —SCN or OAc. In some embodiments, two X groups together with the boron atom to which they are attached form a 6-membered ring having the following structure:

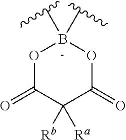

wherein R^{a} and R^{b} are each independently H, CH₃, CH₂CH₃ or an optionally substituted C₁₋₈ alkyl, wherein the substituent for the alkyl is selected from C₁₋₄alkoxy, benzyl, phenyl, or C₁₋₄alkylC(O)O—. In a preferred embodiment, R^{a} and R^{b} are H.

The oxidizing agents are those that are capable of cleaving carbon-boron bond to form an alcohol and a borate ester. Preferred oxidizing agents include peroxide, such as hydrogen peroxide, benzoyl peroxide, t-butyl peroxide, lauroyl peroxide or sodium perborate. In a preferred embodiment, the oxidizing agent is H₂O₂ or sodium perborate.

The borohydride salts are either commercially available (e.g., Sigma Aldrich) or readily can be prepared by reacting a sodium salt with diborane (see Hui, Inorganic Chemistry 1980, 19, 3185-6). Exemplary borohydride salts include M⁺B⁻H₄, M⁺B⁻H₃CN, M⁺B⁻H(OAc)₃, M⁺B⁻H(Et)₃ and M⁺B⁻H(s-butyl)₃, where M⁺ is Na⁺ or K⁺.

The synthesis of high molecular weight aliphatic alcohols can be carried out in situ by reacting borohydride salts, high molecular weight α-olefins and malonic acid or its derivatives followed by an oxidation process. Alternatively, the synthesis can be performed by reacting a high molecular weight α-olefin with a compound of formula (I) followed by an oxidation process.

The hydroboration reactions can be carried out at a temperature from about 20° C. to about 200° C. In some embodiments, the reactions can be carried out at a temperature range selected from 20-100, 50-160, 70-150, 90-180, 80-200 or 150-250° C. For example, the reactions can be carried out at a temperature selected from 20, 30, 40, 50, 60, 70, 80, 90, 100, 110, 120, 130, 140, 150, 160, 170, 180, 190 or 200° C.

Various solvents can be used in the hydroboration-oxidation reactions. Exemplary solvents include, but are not limited to, tetrahydrofuran, diglyme, diisopropyl ether, t-butyl methyl ether, diethyl ether, acetonitrile, dimethoxyethane (DME), alkanes such as hexanes or petroleum ether, benzene, toluene, xylene and combinations thereof.

The high molecular weight primary aliphatic alcohols can be separated and/or purified from unreacted starting materials or side products using work-up procedures known in the art, which include, flash column chromatography, liquid chromatography, HPLC, recrystallization and precipitation. The high molecular weight primary aliphatic alcohols obtained exhibit a high regioselectivity. For example, the regioselectivity of the alcohols is greater than 97%. In some embodiments, the regioselectivity is greater than 98%. In a preferred embodiment, the regioselectivity is greater than 99%. In certain instances, the high molecular weight primary aliphatic alcohols have a regioselectivity of greater than 99.1, 99.2, 99.3, 99.4, 99.5, 99.6, 99.7, 99.8 or 99.9 percent. The regioselectivity of the reactions can also be controlled by selecting appropriate X groups. In one embodiment, X is t-butyl, —OC(O)-t-Bu or dimethylphenylmethyl. In another embodiment, the regioselectivity of the reactions can be adjusted by varying R^{a} and R^{b} groups.

### EXAMPLES

All glassware, syringes, cannulas and needles etc. were dried in an oven at >100° C. overnight.

### Example 1

This example illustrates the synthesis of high molecular weight aliphatic primary alcohols having 26-28 carbon atoms.

Dry 100 mL and 500 mL flasks, with a canula connecting them and magnetic stir bars in each were purged with N₂. About 6.86 g of malonic acid was added to the 100 mL flask. About 2.50 g of NaBH₄ and 30.00-40.00 g of a mixture of C26-28 alpha olefins was added to the 500 mL flask. A dry syringe, with a dry needle was used to add 40 mL of anhydrous diglyme to the 100 mL flask and 60 mL of anhydrous diglyme to the 500 mL flask. Both flasks were stirred. A clear solution formed in the 100 mL flask while a white slurry formed in the 500 mL flask. The malonic acid solution was transferred drop-wise, by canula, with N₂ over 45 minutes. A condenser was attached to the 500 mL flask and the solution was heated to 160° C. for at least 12 hours with an oil bath and constant stirring. A bright yellow-orange color began to appear after 20-30 minutes of heating. The solution was allowed to cool slowly to about 60-70° C., and 40 mL of about 3 M NaOH was added slowly. After bubbling ceased, 10 mL of 30% H₂O₂ was added drop-wise. The solution became a transparent, bright orange color with the addition of NaOH, and a clear, layered solution was formed with the addition of H₂O₂. The condenser was reattached and the solution was heated at 70-80° C. for an additional 2 hours. The solution was then allowed to cool slightly to 60-70° C. and acidified with 3 M HCl to a pH of about 3. The hot solution was immediately transferred to a beaker, where it was allowed to solidify. The resulting mixture was vacuum filtered and rinsed with distilled H₂O. The collected solid was reheated slowly, until there were no large clumps, and rinsed with distilled H₂O. This was repeated for additional purity. The product was allowed to air-dry overnight to remove excess water.

### Example 2

This example illustrates the synthesis of a high molecular weight aliphatic primary alcohol containing 18 carbon atoms.

Dry 250 mL and 500 mL flasks with a canula connecting them and magnetic stir bars in each were purged with N₂ for 15 minutes. About 12.37 g of malonic acid was added to the 250 mL flask. About 4.50 g of NaBH₄ and 30 g of C18 alpha olefin (Chevron Phillips) was added to the 500 mL flask. A dry syringe with a dry needle was used to add 80 mL of anhydrous diglyme to the 250 mL flask and 120 mL of anhydrous diglyme to the 500 mL flask. The malonic acid solution was transferred drop-wise, by canula, with N₂ over 90 minutes. A condenser was attached to the 500 mL flask and the solution was heated to 160° C. for 70 minutes. A bright yellow-orange color began to appear after 15 minutes of heating. The solution was allowed to cool to about 60-70° C. and 40 mL of about 3 M NaOH was added slowly. After bubbling ceased, about 20 mL of 30% H₂O₂ was added dropwise. The solution became a transparent, bright orange color with the addition of NaOH, and a clear, layered solution was formed with the addition of H₂O₂. The condenser was reattached and the solution was heated at 70-80° C. for an additional 2 hours. The solution was then allowed to cool slightly to 60-70° C. and acidified with about 60 mL of about 3 M HCl (until blue litmus paper turned red). The hot solution was transferred to a beaker where it was allowed to solidify (30-60 minutes). The resulting solid was rinsed with 500 mL of distilled water and vacuumed filtered. The product was allowed to air-dry overnight to remove excess water. GC analysis of the raw product showed 70% 1-octadecanol (retention time compared to a 1-octadecanol standard). Recrystallization in n-hexane, n-octane, or n-decane yielded product in >97% purity (by GC and H-NMR).

### Example 3

This example illustrates the synthesis of a high molecular weight aliphatic primary alcohol containing 18 carbon atoms using ethylmalonic acid.

Dry 50 mL and 100 mL flasks with a canula connecting them and magnetic stir bars in each were purged with N₂ for 15 minutes. About 1.00 g of ethylmalonic acid (Sigma Aldrich) was added to the 50 mL flask. About 0.286 g of NaBH₄ and 1.91 g of C18 alpha olefin (Chevron Phillips) was added to the 100 mL flask. A dry syringe with a dry needle was used to add 11 mL of anhydrous digylme to the 50 mL flask and 20 mL of anhydrous diglyme to the 100 mL flask. The ethylmalonic acid solution was transferred drop-wise, by canula, with N₂ over 90 minutes. A condenser was attached to the 100 mL flask and the solution was heated to 160° C. for 19 hours. An off-white color began to appear after 90 minutes of heating. After heating overnight, the solution had a yellow color. The solution was allowed to cool to about 60-70° C. and 4.0 mL of about 3 M NaOH was added slowly. After bubbling ceased, about 2 mL of 30% H₂O₂ was added. The solution became a transparent, bright yellow color with the addition of NaOH, and a clear, layered solution was formed with the addition of H₂O₂. The condenser was reattached and the solution was heated at 70-80° C. for an additional 2 hours. The solution was then allowed to cool slightly to 60-70° C. and acidified with about 6 mL of about 3 M HCl (until blue litmus paper turned red). The hot solution was transferred to a beaker where it was allowed to solidify (30-60 minutes). The resulting solid was rinsed with 50 mL of distilled water and vacuumed filtered. The product was allowed to air-dry overnight to remove excess water. GC analysis of the raw product showed 66% 1-octadecanol (retention time compared to a 1-octadecanol standard). Recrystallization in n-hexane, n-octane, or n-decane yielded product in 99.9% purity (by GC).

### Example 4

This example illustrates the synthesis of a high molecular weight aliphatic primary alcohol containing 18 carbon atoms using dimethylmalonic acid.

Dry 50 mL and 100 mL flasks with a canula connecting them and magnetic stir bars in each were purged with N₂ for 15 minutes. About 1.00 g of dimethylmalonic acid (Sigma Aldrich) was added to the 50 mL flask. About 0.286 g of NaBH₄ and 1.9 g of C18 alpha olefin (Chevron Phillips) was added to the 100 mL flask. A dry syringe with a dry needle was used to add about 11 mL of anhydrous digylme to the 50 mL flask and about 20 mL of anhydrous diglyme to the 100 mL flask. The dimethylmalonic acid solution was transferred drop-wise, by canula, with N₂ over 45 minutes. During the canula transfer, the 100 mL flask was heated to 50-60° C. A condenser was attached to the 100 mL flask and the solution was heated to 160° C. for 90 minutes. The reaction solution remained a white color throughout the 90-minute heating. The solution was allowed to cool to about 60-70° C. and 5 mL of about 3 M NaOH was added slowly. The resulting solution turned a yellow color. Next, 2 mL of 30% H₂O₂ was added. The solution bubbled and white foam formed on the top of the solution. A clear, layered solution was formed with the addition of H₂O₂. The condenser was reattached and the solution was heated at 70-80° C. for an additional 2 hours. The solution was then allowed to cool slightly to 60-70° C. and acidified with about 10 mL of about 3 M HCl (until blue litmus paper turned red). The hot solution was transferred to a beaker where it was allowed to solidify (30-60 minutes). The resulting solid was rinsed with 50 mL of distilled water and vacuumed filtered. The product was allowed to air-dry overnight to remove excess water. GC analysis of the raw product showed 75% 1-octadecanol (retention time compared to a 1-octadecanol standard). Recrystallization in n-hexane, n-octane, or n-decane yielded product in 99.9% purity (by GC).

### Example 5

This example illustrates the synthesis of a high molecular weight aliphatic primary alcohol containing 18 carbon atoms using methylmalonic acid.

Dry 50 mL and 100 mL flasks with a canula connecting them and magnetic stir bars in each were purged with N₂ for 15 minutes. About 0.997 of methylmalonic acid (Sigma Aldrich) was added to the 50 mL flask. About 0.342 g of NaBH₄ and 2.158 g of C18 alpha olefin (Chevron Phillips) was added to the 100 mL flask. A dry syringe with a dry needle was used to add 6 mL of anhydrous digylme to the 50 mL flask and 8 mL of anhydrous diglyme to the 100 mL flask. The methylmalonic acid solution was transferred drop-wise, by canula, with N₂ over 50 minutes. During the canula transfer, the 100 mL flask was heated to 50-60° C. A condenser was attached to the 100 mL flask and the solution was heated to 160° C. for 90 minutes. The reaction solution remained a white color throughout the 90-minute heating. The solution was allowed to cool to about 60-70° C. and 6 mL of about 3 M NaOH was added slowly. The resulting solution turned an orange color. Next, 2 mL of 30% H₂O₂ was added. The solution bubbled and white foam formed on the top of the solution. The condenser was reattached and the solution was heated at 70-80° C. for an additional 2 hours. The solution was then allowed to cool slightly to 60-70° C. and acidified with about 11 mL of about 3 M HCl (until blue litmus paper turned red). The hot solution was transferred to a beaker where it was allowed to solidify overnight. The resulting solid was rinsed with 50 mL of distilled water and vacuumed filtered. The product was allowed to air-dry overnight to remove excess water. GC analysis of the raw product showed 76% 1-octadecanol (retention time compared to a 1-octadecanol standard). Recrystallization in n-hexane, n-octane, or n-decane yielded product in 99.9% purity (by GC).

### Example 6

This example illustrates the synthesis of a high molecular weight aliphatic primary alcohol containing 30 or more carbon atoms.

Dry 250 mL and 500 mL flasks with a canula connecting them and magnetic stir bars in each were purged with N₂ for 15 minutes. About 6.864 g of malonic acid was added to the 250 mL flask. About 2.511 g of NaBH₄ and 28.457 g of C30+ alpha olefin (Chevron Phillips) was added to the 500 mL flask. This starting material contains a mixture of alpha olefins from about C22 to about C40. A dry syringe with a dry needle was used to add 80 mL of anhydrous digylme to the 250 mL flask and 120 mL of anhydrous diglyme to the 500 mL flask. The 500 mL flask was heated to 80° C. The malonic acid solution was transferred drop-wise, by canula, with N₂ over 30-35 minutes. A condenser was attached to the 500 mL flask and the solution was heated to 160° C. for 19 hours. A yellow color began to appear within 15-20 minutes of heating. The solution was allowed to cool to about 60-70° C. and 40 mL of about 3 M NaOH was added slowly after which a solid formed. The solid was heated to 85-90° C. and about 10 mL of 30% H₂O₂ was added dropwise. The solution foamed and a clear, layered solution was formed with the addition of H₂O₂. The condenser was reattached and the solution was heated at 75-80° C. for an additional 2 hours. Next, the solution was acidified with about 60 mL of about 3 M HCl (until blue litmus paper turned red). The hot solution was transferred to a beaker where it was allowed to solidify. The resulting solid was rinsed 6-10 times with 250 mL of distilled water and vacuumed filtered. The product was allowed to air-dry overnight to remove excess water. Recrystallization in n-hexane yielded a mixture of primary alcohols as determined by GC. NMR confirmed the formation of the primary alcohols.

1. A method for the preparation of a high molecular weight aliphatic primary alcohol, said method comprising:
(a) contacting a high molecular weight α-olefin with a borohydride salt of the formula: M⁺B⁻HₙX₄₋ₙ and a malonic acid having the formula: HOC(O)—C(R^{a})(R^{b})—C(O)OH under conditions sufficient to form an intermediate product; and
(b) contacting the intermediate product with an oxidizing agent to form the high molecular weight aliphatic primary alcohol;
wherein
M⁺ is a cation selected from Li⁺, Na⁺, K⁺, Cs⁺ or N⁺R^{c}₄, wherein R^{c} is Et, Bu, benzyl or C₈₋₂₆ alkyl;
the subscript n is an integer from 1 to 4;
each X is independently H, —CN, or —OC(O)C₁₋₈ alkyl; and
R^{a} and R^{b} are each independently selected from the group consisting of H, CH₃, CH₂CH₃, i-Pr, i-Bu or t-Bu.

2. The method of claim 1, wherein said step (a) comprises: (i) contacting a high molecular weight α-olefin with a borohydride salt of the formula: M⁺B⁻HₙX₄₋ₙ to form a reaction mixture; and (ii) contacting said reaction mixture with a malonic acid having the formula: HOC(O)—C(R^{a})(R^{b})—C(O)OH under conditions sufficient to form the intermediate product.

3. The method of claim 1, wherein said step (a) comprises: (i) contacting a borohydride salt of the formula: M⁺B⁻HₙX₄₋ₙ with a malonic acid having the formula: HOC(O)—C(R^{a})(R^{b})—C(O)OH to form a reaction mixture; and (ii) contacting a high molecular weight α-olefin with said reaction mixture under conditions sufficient to form the intermediate product.

4. A method for the preparation of a high molecular weight aliphatic primary alcohol, said method comprising:
(a) contacting a high molecular weight α-olefin with a borohydride salt of the formula: 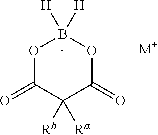
under conditions sufficient to form an intermediate product; and
(b) contacting the intermediate product with an oxidizing agent to form the high molecular weight aliphatic primary alcohol;
Wherein
M⁺ is a cation selected from Li⁺, Na⁺, K⁺, Cs⁺ or N⁺R^{c}₄, wherein R^{c} is Et, Bu, benzyl or C₈₋₂₆ alkyl;
the subscript n is an integer from 1 to 4; and
R^{a} and R^{b} are each independently selected from the group consisting of H, CH₃ and CH₂CH₃.

5. The method of claim 1, wherein the oxidizing agent is H₂O₂ or sodium perborate.

6. The method of claim 1, wherein the high molecular weight aliphatic primary alcohol has a regioselectivity of greater than 99%.

7. The method of claim 1, further comprising:
(c) separating said high molecular weight aliphatic primary alcohol.

8. The method of claim 1, wherein said contacting is in the presence of a solvent selected from the group consisting of tetrahydrofuran, diglyme, diisopropyl ether, t-butyl methyl ether, diethyl ether, acetonitrile, alkanes, benzene, toluene, xylene and combinations thereof.

9. The method of claim 1, wherein said conditions comprise mixing at an elevated temperature of from 35° C. to 200° C.

10. The method of claim 1, wherein said high molecular weight α-olefin is present as a mixture of two or more high molecular weight α-olefins.

11. The method of claim 1, wherein said high molecular weight α-olefin is present as a mixture of greater than C₃₀ high molecular weight α-olefins.

12. The method of claim 1, wherein said high molecular weight α-olefin is present as a mixture of C₂₆ to C₃₀ high molecular weight α-olefins.

13. The method of claim 1, wherein said high molecular weight α-olefin is present as a mixture of C₁₂ to C₁₈ high molecular weight α-olefins.

14. The method of claim 1, wherein said high molecular weight α-olefin is a C₁₂ to C₃₀ α-olefin.

15. The method of claim 1, wherein said high molecular weight α-olefin is greater than C₃₀ high molecular weight α-olefin.

16. The method of claim 1, wherein R^{a} and R^{b} are H.

17. The method of claim 16, wherein M⁺ is Na⁺.

18. The method of claim 1, wherein the high molecular weight α-olefin is unbranched, branched, or a mixture thereof.

19. The method of claim 1, wherein n is 3 and X is CN.

20. The method of claim 1, wherein n is 1 and X is OAc or —O(CO)-t-Bu.

21. The method of claim 1, wherein M⁺ is (n-Bu)₄N⁺.
